(19) United States
(12) Patent Application Publication
(10) Pub. No.: US 2025/0276103 A1
(43) Pub. Date: Sep. 4, 2025

(54) VEHICLE DISINFECTION CONTROL METHOD, SYSTEM AND ELECTRONIC DEVICE

(71) Applicants: Futaijing Precision Electronics (Yantai) Co., Ltd., Yantai (CN); HON HAI PRECISION INDUSTRY CO., LTD., New Taipei (TW)

(72) Inventor: CHUN-YUAN CHEN, New Taipei (TW)

(21) Appl. No.: 18/629,908

(22) Filed: Apr. 8, 2024

(30) Foreign Application Priority Data
Mar. 1, 2024 (CN) 202410232743.5

(51) Int. Cl. A61L 2/10; A61L 2/24; A61L 2/26

(52) U.S. Cl. CPC A61L 2/10; A61L 2/24; A61L 2/26; A61L 2202/11; A61L 2202/14; A61L 2202/25

(57) ABSTRACT

A vehicle disinfection control method comprises enabling a living biological detection device in response to disinfection instructions, obtaining a first operating state of the living biological detection device and first living detection information detected by the living biological detection device, wherein the first living detection information illustrates whether a living organism is in a vehicle, enabling a disinfection device in the vehicle when the first operating state is normal, and the first living detection information illustrates that no living organism is in the vehicle, and keeping the disinfection device in a closed state when the first operating state is abnormal, or the first living detection information illustrates that the living organism is in the vehicle. A vehicle disinfection control system and an electronic device are also disclosed.

### FIELD

The subject matter herein generally relates to vehicle disinfection field.

### BACKGROUND

More and more consumers buy vehicles with a development of vehicle field. The frequency of using vehicles is also increasing.

In recent years, different viruses have emerged one after another, people pay more and more attention to health. Interior of the vehicle is a confined space and is a place that the virus may spread. People may use alcohol spray guns to spray alcohol. However, spraying alcohol has problems such as incomplete disinfection, or alcohol may have adverse reactions to the human body.

### BRIEF DESCRIPTION OF THE DRAWINGS

Implementations of the present disclosure will now be described, by way of embodiments, with reference to the attached figures.

FIG. 1 is a flowchart illustrating an embodiment of a vehicle disinfection control method according to the present disclosure.

FIG. 2 is a diagram showing an embodiment of a vehicle disinfection control system according to the present disclosure.

FIG. 3 is a diagram showing an embodiment of a vehicle according to the present disclosure.

FIG. 4 is a diagram showing modules illustrating an electronic device.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to better illustrate details and features of the present disclosure. It should be noted that references to “an” or “one” embodiment in this disclosure are not necessarily to the same embodiment, and such references mean “at least one”.

Several definitions that apply throughout this disclosure will now be presented.

The connection can be such that the objects are permanently connected or releasably connected. The term “comprising,” when utilized, means “including, but not necessarily limited to;” it specifically indicates open-ended inclusion or membership in the so-described combination, group, series, and the like.

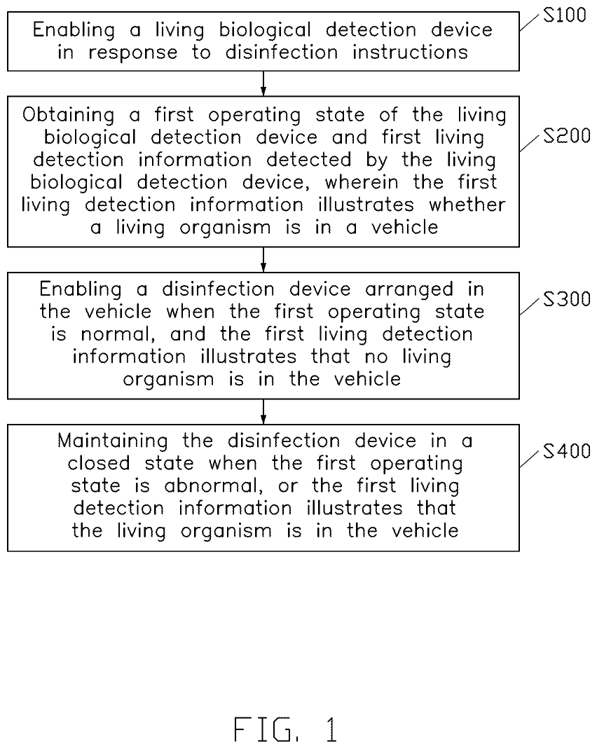

FIG. 1 illustrates one exemplary embodiment of a vehicle disinfection control method. The flowchart presents an exemplary embodiment of the method. The exemplary method is provided by way of example, as there are a variety of ways to carry out the method. Each block shown in FIG. 1 may represent one or more processes, methods, or subroutines, carried out in the example method. Furthermore, the illustrated order of blocks is illustrative only and the order of the blocks can change. Additional blocks can be added, or fewer blocks may be utilized, without departing from this disclosure.

The vehicle disinfection control method can be applied in a vehicle disinfection control system. The vehicle disinfection control system can be installed in a vehicle, the vehicle can be household vehicles, commercial vehicles, engineering vehicles, etc.

In block S100, a living biological detection device is enabled in response to disinfection instructions.

In the embodiment, the living biological detection device may include camera device and infrared detection device. Two camera devices can be installed on each of rear-view mirrors of the vehicle, and/or four infrared detection devices can be installed on each of the four doors, the camera device and the infrared detection device detect whether a living organism exists in the vehicle.

In some embodiments, the vehicle disinfection control system obtains a driving state of the vehicle, the driving state of the vehicle includes a driving state or a parking state. The living biological detection device is enabled when the driving state of the vehicle is in the parking state. Specially, the living biological detection device in the parking state means that the engine of the vehicle stops working. The living biological detection device is kept in the closed state when the driving state of the vehicle is in the driving state. The living biological detection device cannot be enabled when the vehicle is in the parking state, and the living biological detection device in the closed state means the living biological detection device is not in a working state.

In one embodiment, a user can enable the vehicle disinfection control system according to disinfection needs. The vehicle disinfection control system can generate the disinfection instructions. Or the user sets disinfection conditions in the vehicle disinfection control system, and when the disinfection conditions are met, the vehicle disinfection control system generates the disinfection instructions. The disinfection conditions can be that the vehicle is not in use for a longer period than a preset time, and the preset time can be 10 hours, 15 hours etc.

In block S200, a first operating state of the living biological detection device and first living detection information detected by the living biological detection device are obtained, the first living detection information illustrates whether a living organism is in a vehicle.

In one embodiment, a single disinfection duration time, a disinfection opening time, and a living detection duration time preset by the user in the vehicle are obtained. The vehicle disinfection control system provides screens, the user can set one or more of the single disinfection duration time, the disinfection opening time, and the living detection duration time by the screens. For example, the user can set the living detection duration time is 3 minutes. The camera device and/or the infrared detection device can detect whether the living organism is in the vehicle, when the current driving state of the vehicle is in the parking state.

In block S300, a disinfection device in the vehicle is enabled when the first operating state is normal, and the first living detection information illustrates that no living organism is in the vehicle.

In one embodiment, the user can set the single disinfection duration time and the disinfection opening time. For example, the user can set the single disinfection duration time is 5 minutes and the disinfection opening time is 10:00 PM. When the first operating state is normal, and the first living detection information illustrates that no living organism is in the vehicle, the disinfection device is enabled at the disinfection opening time and disinfects the vehicle within the single disinfection duration time. For example, the disinfection device is enabled at 10:00 PM and a disinfection time of the disinfection device is 5 minutes.

In one embodiment, the disinfection device includes ultraviolet disinfection lamps. The ultraviolet disinfection lamps can be installed at passenger handle of the door, or at middle positions of the vehicle roof respond to four seats. The ultraviolet disinfection lamps can disinfect each area of the vehicle. In some embodiments, the disinfection device also includes a portable electronic spray disinfection machine, the portable electronic spray disinfection machine can be loaded with alcohol or other liquids for disinfection.

Specially, the ultraviolet disinfection lamps are enabled in response to light intensity adjustment instructions of the ultraviolet disinfection lamps. For example, the user can also set light intensity adjustment information of the ultraviolet disinfection lamp to achieve adjusting the light intensity of the ultraviolet disinfection lamp, the light intensity adjustment information includes a first level information, a second level information and a third level information.

In one embodiment, a wavelength of the first level information is in a range between 320 nm-400 nm, a wavelength of the second level information is in a range between 280 nm-320 nm, a wavelength of the third level information is in a range between 200 nm-280 nm.

In one embodiment, a cumulative disinfection duration time of the ultraviolet disinfection lamps is obtained. Replacement device information of the ultraviolet disinfection lamps is generated when the cumulative disinfection duration time is larger than a maximum service life of the ultraviolet disinfection lamps, the replacement device information illustrates whether the ultraviolet disinfection lamps need to be replaced.

In the embodiment, the vehicle disinfection control system obtains the cumulative disinfection duration time corresponding to each ultraviolet disinfection lamp and generates the replacement device information corresponding to each ultraviolet disinfection lamp, when the cumulative disinfection duration time is larger than the maximum service life of the ultraviolet disinfection lamps. Therefore, when the ultraviolet disinfection lamp is replaced in time, a problem of incomplete disinfection can be avoided.

In some embodiments, a second operating state of the living biological detection device is obtained, a second living detection information detected by the living biological detection device is obtained, and a third operating state of the disinfection device is obtained, in the process of disinfection of the vehicle by the disinfection device, the second living detection information illustrates whether the living organism is in the vehicle during the process of disinfection of the vehicle by the disinfection device.

The disinfection device is controlled in a closed state, when the living organism is in the vehicle, the second operating state is abnormal, or the third operating state is abnormal. In the embodiment, the disinfection device is electrically connected to a battery in the vehicle. The disinfection device in the close state means that the disinfection device is not electrically connected with the control device.

In one embodiment, the vehicle disinfection control can also obtain a first power of the battery and a second power consumed by the disinfection device during the single disinfection duration time, and when the first power is less than the second power, a reminder message of insufficient power is displayed on the screens to remind the user to disinfect after a period of time. In other embodiments, the vehicle disinfection control system can also display the reminder message of insufficient power through a horn.

In one embodiment, when the disinfection device disinfects the vehicle, the first living detection information may be wrong. For example, an adult and a pet are in the vehicle, when the vehicle is in the parking state, the living biological detection device detects the adult getting out of the vehicle, but the pet is in the trunk of the vehicle. The living biological detection device does not detect the pet, the first living detection information illustrates that no living organism is in the vehicle. The disinfection device is enabled to disinfect the vehicle, and the pet runs to the seats of the vehicle, the second living detection information illustrates the living organism (the pet) is in the vehicle, the disinfection device will be closed to prevent the disinfection device from harming the pet.

In other embodiments, when the disinfection device disinfects the vehicle, if the second operating state of the living biological detection device is abnormal, and the living biological detection device fails. For example, the living biological detection device can not work normally, it is necessary to control the disinfection device to close, when the pet is in the vehicle, a situation that the disinfection device is not closed in time and harms the pet is avoided.

In another embodiment, when the disinfection device disinfects the vehicle, if the third operating state of the living biological detection device is abnormal, and the disinfection device fails. For example, when the disinfection device is the ultraviolet disinfection lamps, one ultraviolet sterilization lamp has been flashing and cannot work normally, the disinfection device must be disconnected from a power state to avoid complete damage to the ultraviolet sterilization lamps.

In block S400, the disinfection device is kept in the closed state when the first operating state is abnormal, or the first living detection information illustrates that the living organism is in the vehicle.

In some embodiments, when the operating state is abnormal, the living biological detection device is breakdown. For example, when the living biological detection device is infrared detection devices, one or two of the four infrared detection devices fail, the first living detection information detected by the non-malfunctioning infrared detection devices illustrates that no living organism is in the vehicle, in fact, one or more living organisms are in the vehicle. The first living detection information is wrong, the disinfection device should be controlled by the vehicle disinfection control system in the closed state. The living organism is protected from harm by the disinfection device.

The single disinfection duration time, the disinfection opening time, and the living detection duration time can be preset by the user in the vehicle. When the vehicle is in the parking state, the living biological detection device is enabled to detect whether the living organism is in the vehicle. On the one hand, when the living biological detection device detects the living organism is in the vehicle, or when the living biological detection device is abnormal, the disinfection device is kept in the closed state. When the living organism is in the vehicle, the disinfection device is mistakenly enabled to harm the living organism. At the same time, when the disinfection device disinfects the vehicle, an operating status of the living biological detection device and an operating status of the disinfection device are obtained to determine whether the disinfection device is closed in time.

On the other hand, when the living biological detection device detects that no living organism is in the vehicle, and the living biological detection device is normal, the disinfection device is enabled to disinfect the vehicle. At the same time, when the disinfection device is the ultraviolet disinfection lamps, the user can also set the appropriate light intensity adjustment information to disinfect the vehicle. When the cumulative disinfection duration time is larger than the maximum service life of the ultraviolet disinfection lamps, the user is reminded to replace the ultraviolet disinfection lamps in time, when the ultraviolet disinfection lamps use long, a disinfection effect of the ultraviolet disinfection lamps can be avoided.

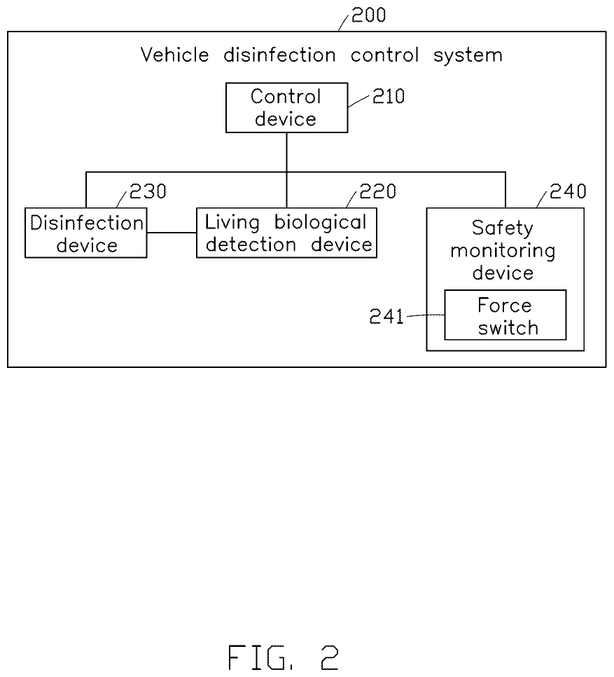

Please refer to FIG. 2, FIG. 2 shows the vehicle disinfection control system 200. The vehicle disinfection control system 200 is installed in the vehicle. The vehicle disinfection control system 200 includes a control device 210, the living biological detection device 220, the disinfection device 230 and a safety monitoring device 240. The living biological detection device 220, the disinfection device 230 and the safety monitoring device 240 are communicated with the control device 210, the safety monitoring device 240 is communicated with the living biological detection device 220.

In one embodiment, the control device 210 enables the living biological detection device 220 in response to the disinfection instructions. The living biological detection device 220 detects whether the living organism is in the vehicle and generates the first living detection information. The safety monitoring device 240 obtains the first operating state of the living biological detection device 220. The disinfection device 230 disinfects the vehicle when the first operating state is normal, and the first living detection information illustrates that no living organism is in the vehicle.

In one embodiment, when the first operating state is abnormal, or the first living detection information illustrates that the living organism is in the vehicle, the safety monitoring device 240 sends stopping disinfection instructions to the control device 210. The control device 210 controls the disinfection device 230 is in the closed state. In the embodiment, the safety monitoring device 240 includes a force switch 241. When the disinfection device 230 is disinfecting the vehicle, the user can turn off the disinfection device 230 by the force switch 241.

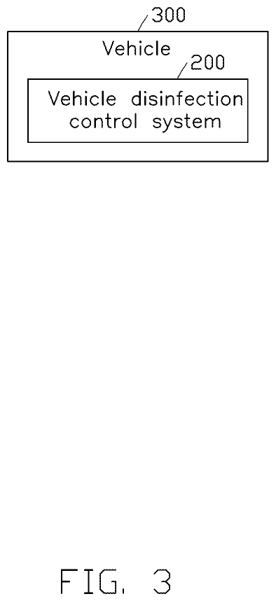

In one embodiment, as shown in FIG. 3, the vehicle 300 includes the vehicle disinfection control system.

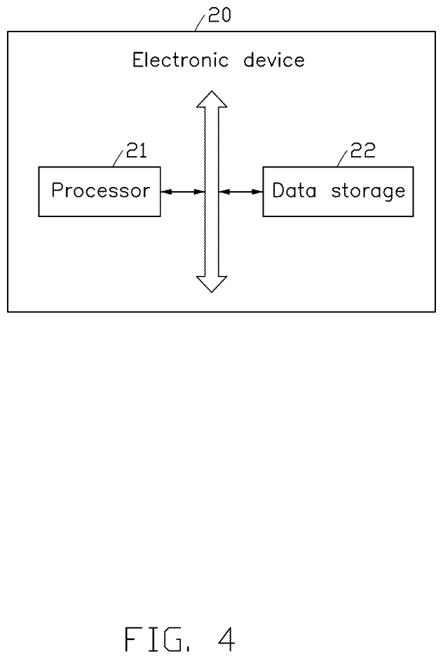

As shown in FIG. 4, one exemplary embodiment of an electronic device 20 comprises at least one processor 21 and a data storage 22. The data storage 22 stores one or more programs which can be executed by the at least one processor 21. The data storage 22 is used to store instructions, and the processor 21 is used to call up instructions from the data storage 22, so that the electronic device 20 performs the steps of the vehicle disinfection control method in the above embodiment.

In one embodiment, a non-transitory storage medium recording instructions is disclosed. When the recorded computer instructions are executed by a processor of an electronic device 20, the electronic device 20 can perform the method.

The embodiments shown and described above are only examples. Many details known in the field are neither shown nor described. Even though numerous characteristics and advantages of the present technology have been set forth in the foregoing description, together with details of the structure and function of the present disclosure, the disclosure is illustrative only, and changes may be made in the detail, including in matters of shape, size, and arrangement of the parts within the principles of the present disclosure, up to and including the full extent established by the broad general meaning of the terms used in the claims. It will therefore be appreciated that the embodiments described above may be modified within the scope of the claims.

What is claimed is:

1. A vehicle disinfection control method comprising:
enabling a living biological detection device in response to disinfection instructions;
obtaining a first operating state of the living biological detection device and first living detection information detected by the living biological detection device, wherein the first living detection information illustrates whether a living organism is in a vehicle;
enabling a disinfection device arranged in the vehicle when the first operating state is normal, and the first living detection information illustrates that no living organism is in the vehicle; and
maintaining the disinfection device in a closed state when the first operating state is abnormal, or the first living detection information illustrates that the living organism is in the vehicle.

2. The vehicle disinfection control method of claim 1, wherein after enabling the disinfection device arranged in the vehicle, the vehicle disinfection control method further comprises:
obtaining a second operating state of the living biological detection device, second living detection information detected by the living biological detection device, and a third operating state of the disinfection device in a process of disinfection of the vehicle by the disinfection device, wherein the second living detection information illustrates whether the living organism is in the vehicle during the process of disinfection of the vehicle; and
controlling the disinfection device in the closed state when the second living detection information illustrates that the living organism is in the vehicle, the second operating state is abnormal, or the third operating state is abnormal.

3. The vehicle disinfection control method of claim 1, wherein the disinfection device comprises ultraviolet disinfection lamps; enabling the disinfection device in the vehicle comprises:
enabling and configuring the ultraviolet disinfection lamps in response to light intensity adjustment instructions of the ultraviolet disinfection lamps.

4. The vehicle disinfection control method of claim 3, further comprising:
obtaining a cumulative disinfection duration time of the ultraviolet disinfection lamps; and
generating replacement device information of the ultraviolet disinfection lamps when the cumulative disinfection duration time is larger than a maximum service life of the ultraviolet disinfection lamps, wherein the replacement device information illustrates whether the replacement device information can be replaced.

5. The vehicle disinfection control method of claim 1, wherein before enabling the living biological detection device, the vehicle disinfection control method further comprises:
obtaining a driving state of the vehicle, wherein the driving state of the vehicle comprises a driving state or a parking state;
enabling the living biological detection device when the driving state of the vehicle is in the parking state; and
keeping the living biological detection device in the closed state when the driving state of the vehicle is in the driving state.

6. The vehicle disinfection control method of claim 5, wherein after enabling the living biological detection device in response to the disinfection instructions, the vehicle disinfection control method further comprises:
obtaining a single disinfection duration time, a disinfection opening time, and a living detection duration time;
obtaining the first living detection information detected by the living biological detection device comprising:
obtaining the first living detection information detected by the living biological detection device within the living detection duration time;
enabling the disinfection device arranged in the vehicle comprises:
enabling the disinfection device at the disinfection opening time; and
controlling the disinfection device to disinfect the vehicle within the single disinfection duration time.

7. A vehicle disinfection control system, comprising:
a control device, a living biological detection device, a disinfection device, and a safety monitoring device, wherein the control device is communicated with the living biological detection device, the disinfection device, and the safety monitoring device, the living biological detection device is communicated with the safety monitoring device, the control device enables the living biological detection device in response to receiving disinfection instructions; the living biological detection device detects whether a living organism is in a vehicle and generates first living detection information; the safety monitoring device obtains a first operating state of the living biological detection device; the disinfection device disinfects the vehicle when the first operating state is normal, and the first living detection information illustrates that no living organism is in the vehicle.

8. The vehicle disinfection control system of claim 7, wherein the safety monitoring device obtains a second operating state of the living biological detection device, the control device obtains a third operating state of the disinfection device, the living biological detection device generates second living detection information in a process of disinfection of the vehicle by the disinfection device, the second living detection information illustrates whether the living organism is in the vehicle during the process of disinfection of the vehicle; and the control device controls the disinfection device in the closed state when the second living detection information illustrates that the living organism is in the vehicle, the second operating state is abnormal, or the third operating state is abnormal.

9. The vehicle disinfection control system of claim 7, wherein the disinfection device comprises ultraviolet disinfection lamps; the control device enables and configures the ultraviolet disinfection lamps in response to light intensity adjustment instructions of the ultraviolet disinfection lamps.

10. The vehicle disinfection control system of claim 9, wherein the control device obtains a cumulative disinfection duration time of the ultraviolet disinfection lamps and generates replacement device information of the ultraviolet disinfection lamps when the cumulative disinfection duration time is larger than a maximum service life of the ultraviolet disinfection lamps, the replacement device information illustrates that the ultraviolet disinfection lamps need to be replaced.

11. The vehicle disinfection control system of claim 7, wherein a driving state of the vehicle comprises a driving state or a parking state, the control device further obtains the driving state of the vehicle, the control device enables the living biological detection device when the driving state of the vehicle is in the parking state, or keep the living biological detection device in the closed state when the driving state of the vehicle is in the driving state.

12. The vehicle disinfection control system of claim 11, wherein the control device obtains a single disinfection duration time, a disinfection opening time, and a living detection duration time, the control device further obtains the first living detection information detected by the living biological detection device within the living detection duration time, enables the disinfection device at the disinfection opening time, and controls the disinfection device to disinfect the vehicle within the single disinfection duration time.

13. The vehicle disinfection control system of claim 7, wherein the safety monitoring device comprises a force switch, the control device controls the disinfection device is in the closed state by the force switch.

14. An electronic device, comprising:
at least one processor; and
a data storage storing one or more programs which when executed by the at least one processor, cause the at least one processor to:
enable a living biological detection device in response to disinfection instructions;
obtain a first operating state of the living biological detection device and first living detection information detected by the living biological detection device, wherein the first living detection information illustrates whether a living organism is in a vehicle;
enable a disinfection device arranged in the vehicle when the first operating state is normal, and the first living detection information illustrates that no living organism is in the vehicle; and
keep the disinfection device in a closed state when the first operating state is abnormal, or the first living detection information illustrates that the living organism is in the vehicle.

15. The electronic device of claim 14, wherein after the at least one processor enables the disinfection device arranged in the vehicle, the at least one processor is further caused to:
obtain a second operating state of the living biological detection device, second living detection information detected by the living biological detection device, and a third operating state of the disinfection device in a process of disinfection of the vehicle by the disinfection device, wherein the second living detection information illustrates whether the living organism is in the vehicle during the process of disinfection of the vehicle; and
control the disinfection device in the closed state, when the second living detection information illustrates that no living organism is in the vehicle, the second operating state is abnormal, or the third operating state is abnormal.

16. The electronic device of claim 14, wherein the disinfection device comprises ultraviolet disinfection lamps; when the at least one processor enables the disinfection device in the vehicle, the at least one processor is further caused to:
enable and configure the ultraviolet disinfection lamps in response to light intensity adjustment instructions of the ultraviolet disinfection lamps.

17. The electronic device of claim 16, wherein the at least one processor is further caused to:
obtain a cumulative disinfection duration time of the ultraviolet disinfection lamps; and
generate replacement device information of the ultraviolet disinfection lamps when the cumulative disinfection duration time is larger than a maximum service life of the ultraviolet disinfection lamps, wherein the replacement device information illustrates that the ultraviolet disinfection lamps need to be replaced.

18. The electronic device of claim 14, wherein before the at least one processor enables a living biological detection device, the at least one processor is further caused to:
obtain a driving state of the vehicle, wherein the driving state of the vehicle comprises a driving state or a parking state;
enable the living biological detection device when the driving state of the vehicle is in the parking state; and
keep the living biological detection device in the closed state when the driving state of the vehicle is in the driving state.

19. The electronic device of claim 18, wherein after the at least one processor enables a living biological detection device in response to the disinfection instructions, the at least one processor is further caused to:
obtain a single disinfection duration time, a disinfection opening time, and a living detection duration time;
when the at least one processor obtains the first living detection information detected by the living biological detection device, the at least one processor is further caused to:
obtain the first living detection information detected by the living biological detection device within the living detection duration time;
when the at least one processor enables the disinfection device arranged in the vehicle, the at least one processor is further caused to:
enable the disinfection device at the disinfection opening time; and
control the disinfection device to disinfect the vehicle within the single disinfection duration time.
